(19) United States
(12) Patent Application Publication
(10) Pub. No.: US 2009/0197910 A1
(43) Pub. Date: Aug. 6, 2009

(54) NOVEL VIRAL REPLICATION INHIBITORS

(75) Inventors: Johan Neyts, Kessel-Lo (BE); Asish R. Das, Hsinchu 300 (TW); Shih Ching Hung, Hsinchu 300 (TW); Jih Ru Hwu, Hsinchu 300 (TW); Raghunath Singha, Hsinchu 300 (TW)

Correspondence Address: CLARK & ELBING LLP, 101 FEDERAL STREET, BOSTON, MA, 02110, US

(73) Assignees: KATHOLIEKE UNIVERSITEIT LEUVEN, Leuven (BE); NATIONAL TSING HUA UNIVERSITY, Hsinchu 300 (TW)

(21) Appl. No.: 12/299,636

(22) PCT Filed: May 7, 2007
(86) PCT No.: PCT/BE2007/000043

§ 371 Date: Mar. 20, 2009

(30) Foreign Application Priority Data
May 5, 2006 (GB) 0608899.1

(51) Int. Cl. A61K 31/4184; C07D 235/30; C07D 471/04; A61K 31/437; A61P 31/12

(52) U.S. Cl. 514/303; 548/304.7; 546/118; 514/395

(57) ABSTRACT

The present invention relates to a pharmaceutical composition for the treatment or prevention of viral infections comprising as an active principle at least one benzimidazole conjugates derivative having the general formula (I). The invention also relates to processes for the preparation of compounds according to the invention having above mentioned general formula and their use as a medicine or to treat or prevent viral infections.

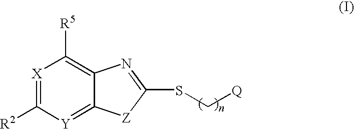

### FIELD OF THE INVENTION

The present invention relates to a series of novel compounds, processes for their preparation, their use to treat or prevent viral infections and their use to manufacture a medicine to treat or prevent viral infections, particularly infections with viruses belonging to the family of the Flaviviridae and more preferably infections with Hepatitis C virus (HCV).

### BACKGROUND OF THE INVENTION

The family of the Flaviviridae consists of 3 genera, the pestiviruses, the flaviviruses and the hepaciviruses and also contains the hepatitis G virus (HGV/GBV-C) that has not yet been assigned to a genus.

The World Health Organization estimates that world-wide 170 million people (3% of the world's population) are chronically infected with HCV. These chronic carriers are at risk of developing cirrhosis and/or liver cancer. In studies with a 10 to 20 year follow-up, cirrhosis developed in 20-30% of the patients, 1 to 5% of whom may develop liver cancer during the next then years. The only treatment option available today is the use of interferon α-2 (or its pegylated from) either alone or combined with ribavirin. However, sustained response is only observed in about 40% of the patients and treatment is associated with serious adverse effects. There is thus an urgent need for potent and selective inhibitors of the replication of the HCV in order to treat infections with HCV. Furthermore, the study of specific inhibitors of HCV replication has been hampered by the fact that it is not possible to propagate HCV (efficiently) in cell culture.

Some benzimidazole derivatives (Giiums, F. et al. Eur. J. Med. Chem. 2003, 38, 473-480 and Andrzejewska, M. et al. Eur. J. Med. Chem. 2002, 37, 973-978) and coumarin derivatives (Ito, C. et al. J. Nat. Prod. 2003, 66, 368-371 and Nam, N.-H. et al. Bioorg. Med. Chem. Lett. 2002, 12, 2345-2348) in the prior art show diverse biological activities with significant clinical value, including their applications to breast cancer, leukaemia, and tumor cells (Demirayak, S. et al. Eur. J. Med. Chem. 2002, 37, 255-260; Garuti, L. et al. Bioorg. Med. Chem. Lett. 2000, 10, 2193-2195; Lukevics, E. et al. Eur. J. Med. Chem. 2001, 36, 507-515; et al. Antonini, I. et al. J. Med. Chem. 1988, 31, 260-264). The prior art also describes some benzimidazole-coumarin conjugates coupled with a thio-linker as in Ahluwalia V. K. et al. Indian Journal of Chemistry, Section B, 1990, 29B(12), 1097-1100 but no antiviral activity has been identified.

Furthermore, in the prior art, benzimidazole derivatives have been shown to possess anti-viral activity (Devivar, R. V. et al. J. Med. Chem. 1994, 37, 2942-2949 and Curini, M. et al. Aust. J. Chem. 2003, 56, 59-60). Examples include a series of non-nubleoside benzimidazoles reported recently by Beaulieu et al with anti-HCV activity (Beaulieu, P. L. et al. Bioorg. Med. Chem. Lett. 2004, 14, 967-971; McKercher, G. et al. Nucleic Acids Res. 2004, 32, 422-431; Beaulieu, P. L. et al. J. Med. Chem. 2004, 47, 6884-6892 and Beaulieu, P. L. et al. Curr. Med. Chem.: Anti-Infect. Agents 2002, 1, 163-176).

The present invention provides for novel compounds which show activity against virusses, more specifically against HCV. There is a clear need in the field for alternative antiviral compounds, furthermore with a good activity vs toxicity profile and this specifically for the virusses of the family of the Flaviviridae, more specifically for Hepatitis C Virus. The prior art does not lead a person skilled in the art to the compounds of the present invention and to their use as antiviral compounds.

### SUMMARY OF THE INVENTION

Herein we disclose our new findings that novel conjugates with a methylenethio linker and the corresponding N-nucleosides exhibited potent inhibition effects HCV. Cytotoxicity was found low for these compounds.

In the present invention, new selective anti-viral compounds are being provided. The compounds are benzimidazole, pyridine-imidazole or purine conjugates and it has been shown that they possess an anti-viral activity. Members of the Flaviviridae family are being inhibited. The present invention demonstrates that the compounds inhibit the replication of HCV. Therefore, these benzimidazole conjugates constitute a new potent class of anti-viral compounds that can be used in the treatment and prevention of viral infections in animals, mammals and humans, more specifically for the treatment and prevention of HCV.

The present invention relates to novel compounds. The invention further relates to novel compounds having anti-viral activity. Most particularly, the invention relates to novel compounds, in a particular embodiment being benzimidazole-coumarine conjugates, which inhibit the replication of viruses of the family of the Flaviviridae and yet more specifically to compounds that inhibit the replication of HCV (Hepatitis C Virus) infections. Present invention furthermore relates to the compounds for use as a medicine and to the use of the compounds as a medicine and more specifically to use the compounds as an anti-viral. The invention also relates to methods for preparation of all such compounds and pharmaceutical compositions comprising them. The invention further relates to the use of said compounds in the manufacture of a medicament useful for the treatment of HCV infections, as well as for treatment of other viral infections, especially other infections with RNA-viruses. The present invention also relates to a method of treatment of viral infections, by using said compounds.

According to a first aspect, the invention relates to novel compounds, which according to the general embodiment of the invention correspond to compounds according to the general formula (I), pharmaceutically acceptable salts, solvates, tautomers, isomers thereof,

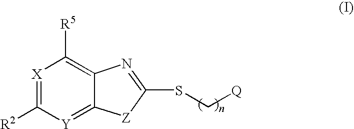

wherein:
X is selected from CR¹ or N;
Y is selected from CR⁶ or N;
Z is selected from NR⁷; O or S;
each of R¹, R², R¹ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8.

A particular embodiment of the invention relates to the compounds according to formula (I) wherein X is CR¹ and Y is N, and thereby the compound has a structure according to formula (II) (forming imidazo[4,5-b]pyridine-heterocyclic ring conjugates):

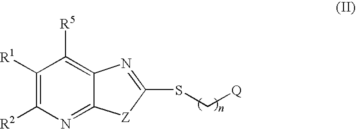

wherein all of R¹, R², R⁵, R⁷, Z, Q and n are as in formula (I).

In another embodiment of formula (I), each of X and Y is N, and thereby the compound has a structure according to formula (III) (forming purine—heterocyclic ring conjugates):

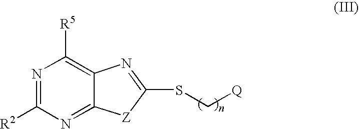

wherein all of R², R⁵, R⁷, Z, Q and n are as in claim 1.

In a particular embodiment of all formulas (I), (II) and (III) herein, Z is NR⁷.
In yet another particular embodiment of formula (I), X is CR¹, Y is CR⁶ and Z is NR⁷, and thereby the compounds of the invention are according to formula (IV), pharmaceutically acceptable salts, solvates, tautomers, or isomers thereof:

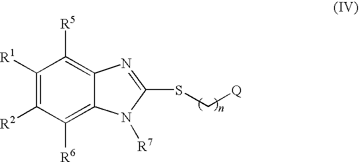

wherein:
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8.

In another embodiment, said substituted aryl or substituted heterocyclic ring are substituted with hydroxy; nitro; alkoxy; a C₁-C₁₆ hydrocarbon group or halogen; wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N.

In yet another particular embodiment of the formulas herein, n is 1.

In another particular embodiment, Q is polycyclic, yet more in particular comprises 2 fused rings. In another particular embodiment of all previous formulas herein, Q is selected from unsubstituted or substituted phenyl, naphthyl, pyridyl and chromen-2-onyl. In a more particular embodiment, Q has a structure according to formula (V):

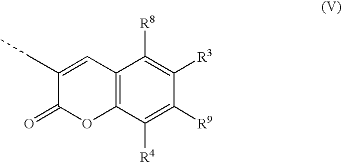

wherein each of R³, R¹, R¹ and R⁹ are independently selected from hydrogen; hydroxy; nitro; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group are optionally replaced by heteroatoms selected from O, S, and N; and n is 1.

In a particular embodiment of this aspect of the invention, each of R¹ and R⁶ are hydrogen. In another particular embodiment, each of R¹ and R² are independently selected from hydrogen; halogen; C₁-C₆ alkyl; methoxy or benzoyl.

In another particular embodiment, R⁷ is a carbohydrate. In yet another particular embodiment, R⁷ is a substituted or unsubstituted glucopyranosyl. In still another more particular embodiment, said substituted or unsubstituted glucopyranosyl has a structure according to the following formula

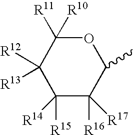

wherein each of R¹⁰, R¹¹, R¹², R¹³, R¹⁴, R¹⁵, R¹⁶ and R¹⁷ can be the same or different and are each independently selected from hydrogen; hydroxy; C₁₋₈ alkyl; C₁₋₈ alkoxy; acetyloxy; halogen; hydroxy-C₁₋₈alkyl and haloC₁₋₈alkyl.

A particular embodiment relates to the benzimidazole-coumarin conjugate compounds, which according to the general embodiment of the invention correspond to compounds according to the general formula (VI), pharmaceutically acceptable salts, solvates, tautomers, isomers thereof,

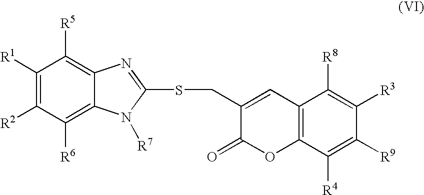

wherein:
each of R¹, R², R³, R⁴, R⁵, R⁶, R⁸ and R⁹ are independently selected from hydrogen; hydroxy; a C₁-C₁₆ hydrocarbon group or halogen; wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;

In a particular embodiment of the present aspect of the invention, each of R⁵, R⁶, R⁸ and R⁹ are hydrogen. In another particular embodiment, each of R¹, R², R³ and R⁴ are independently selected from hydrogen; halogen; C₁-C₆ alkyl; methoxy or benzoyl.

A second aspect of the present invention relates to the compounds according to the formula (I), (II), (III), (IV) and (VI) and embodiments thereof for their use as a medicine, more in particular for their use as an antiviral medicine and for their use in the prevention or treatment of a viral infection.

Another aspect of the present invention relates to the use of compounds according to formula (VII), pharmaceutically acceptable salts, tautomers, and isomers thereof:

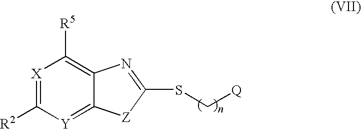

wherein:
X is selected from CR¹ or N;
Y is selected from CR⁶ or N;
Z is selected from NR⁷; O or S;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from hydrogen; substituted or unsubstituted aryl or substituted or unsubstituted heterocyclic ring; and
n is selected from 1 to 8;
   for the manufacture of a medicament for the treatment or prevention of a viral infection in a mammal, provided that Q and R⁷ are not both hydrogen.

This aspect also relates to all embodiments and formulas described herein above.

In a particular embodiment of this aspect, the present invention relates to the use of the compounds according to formula (VII), wherein X is CR¹ and Y is N. In another particular embodiment, the present invention relates to the use of the compounds according to formula (VII), wherein each of X and Y is N. In a particular embodiment, Z is selected from NR⁷. In yet another particular embodiment, the present invention relates to the use of compounds according to formula (VII), wherein X is CR¹, Y is CR⁶ and Z is NR⁷, and thereby the compound has a structure according to the general formula (VIII), pharmaceutically acceptable salts, tautomers, and isomers thereof

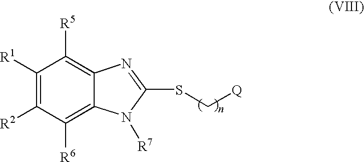

wherein:
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from hydrogen; substituted or unsubstituted aryl or substituted or unsubstituted heterocyclic ring; and
n is selected from 1 to 8;
   for the manufacture of a medicament for the treatment or prevention of a viral infection in a mammal, provided that Q and R⁷ are not both hydrogen.

A particular embodiment relates to the use of the compounds according to formula (I), (II), (III), (IV), (VI), (VII) and (VIII) and embodiments thereof for the manufacture of a medicament, or a pharmaceutical composition having antiviral activity, to treat or prevent viral infections in a mammal.

The present invention further relates to the use of the compounds according to formula (I), (II), (III), (IV), (VI), (VII) and (VIII) above as a medicine and to the use of such compounds in the treatment or prevention of a viral infection in a mammal.

In a particular embodiment, said viral infection is an infection with an RNA-virus, yet more in particular with a virus of the family of the Flaviviridae. Yet more in particular, said viral infection is an infection with Hepatitis C virus (HCV).

In another particular embodiment, said mammal in need of treatment or prevention of a viral infection is a human.

The invention also relates to the use of the compounds according to formula (I), (II), (III), (IV), (VI), (VII) and (VIII) above as a pharmaceutically active ingredient, especially as an inhibitor of the viral replication, more preferably as an inhibitor of the replication of a virus of the family of the Flaviviridae and yet more preferably as an inhibitor of the replication of HCV.

The present invention further relates to a method of treatment of a viral infection in a mammal, including a human, comprising administering to the mammal in need of such treatment (a therapeutically effective amount of) a compound according to formula I above as an active ingredient, optionally in a mixture with at least a pharmaceutically acceptable carrier.

The present invention further relates to a composition for separate, combined or sequential use in the treatment or prophylaxis of anti-viral infections, comprising:

a) one or more of the compounds according to formulas and embodiments above, and
b) one or more compounds effective in the treatment or prophylaxis of viral infections, including Flaviviral enzyme inhibitors; in proportions such as to provide a synergistic effect in the said treatment of prophylaxis.

The invention further relates to methods for the preparation of the compounds according to formulas as detailed above, more particularly to methods for the preparation of the compounds specifically disclosed herein, to pharmaceutical compositions comprising them in admixture with at least a pharmaceutically acceptable carrier, the active ingredient optionally being in a concentration range of about 0.1-100% by weight, and to the use of these derivatives namely as antiviral drugs, more particularly as drugs useful for the treatment of subjects suffering from HCV infection.

The invention also relates to a method for preparing the compounds according to the formulas above and embodiments thereof as described herein. Such a method may essentially comprise the steps of:
a) reacting a substituted or unsubstituted 1,2-diamino-phenyl (or phenylenediamines) with CS₂;
b) coupling the product of (a) with substituted or unsubstituted aryl or heterocyclic ring substituted with halogenalkyl such as 3-chloromethylcoumarins.

For benzimidazole conjugates bearing a carbohydrate, the product of reaction a is first coupled to the carbohydrate, as following:

b′) coupling the product of (a) with completely protected carbohydrate, such as peracetylpyranose;
c′) reacting the product of step (b′) with substituted or unsubstituted aryl or heterocyclic ring substituted with halogenalkyl such as 3-chloromethylcoumarins.
d′) if needed, the carbohydrate protecting groups can be removed, as for example for the deprotection of the carbohydrate acetyl protecting groups, NH₃ in methanol at room temperature for 18 h can be used.

According to a particular embodiment, the present invention relates to compounds selected from the group of compounds specified in the tables 1 and 2 in the application, the pharmaceutically acceptable salts, tautomers, and isomers thereof and their use in a treatment of viral infection or to manufacture a medicament to treat viral infections.

### DETAILED DESCRIPTION OF THE INVENTION

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The terms mentioned herein with prefixes as C₁₋₁₆ can also be used with lower numbers of carbon atoms such as C₁₋₈ or C₁₋₆. If for example the term C₁-C₆ is used, it refers to the presence of between 1 and 6 carbon atoms.

The term “C₁-C₁₆ hydrocarbon group” as used herein refers to C₁-C₁₆ normal, secondary, tertiary unsaturated or saturated, acyclic, cyclic or aromatic hydrocarbons and combinations thereof. The term therefore comprises alkyl, alkenyl, alkynyl, cycloalkyl, cycloalkenyl, cycloalkynyl, aryl, arylalkyl, among others.

The term “carbohydrate” as used herein refers to substituted or unsubstituted cyclic or acyclic sugars such as but not limited to glucofuranoses, glucopyranoses, ribofuranoses and ribopyranose. The carbohydrates can also be substituted with for example amino, carboxy or hydroxy groups.

The term “C₁₋₁₆ alkyl” as used herein refers to C1-C16 normal, secondary, or tertiary unsaturated hydrocarbon. Examples are methyl, ethyl, 1-propyl, 2-propyl, 1-butyl, 2-methyl-1-propyl(1-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, cyclopropyl, cyclobutyl, cyclopentyl and cyclohexyl. In a particular embodiment, the term also includes C₁₋₁₆ halo-alkyls, which is a C₁₋₁₆ alkyl bearing at least one halogen.

As used herein and unless otherwise stated, the term “C₃₋₁₀ cycloalkyl” means a monocyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl.

As used herein and unless otherwise stated, the term “C₃₋₁₀ cycloalkylene” refers to a cyclic hydrocarbon radical of 3-10 carbon atoms, and having two monovalent radical centers derived by the removal of two hydrogen atoms from the same or two different carbon atoms of a parent alkane; i.e. the divalent hydrocarbon radical corresponding to the above defined C₃₋₁₀ cycloalkyl.

The terms “C₂₋₁₈ alkenyl” and “C₃₋₁₀ cycloalkenyl” as used herein is C₂-C₁₈ normal, secondary or tertiary and respectively C₃₋₁₀ cyclic hydrocarbon with at least one site (usually 1 to 3, preferably 1) of unsaturation, i.e. a carbon-carbon, sp2 double bond. Examples include, but are not limited to: ethylene or vinyl (—CH═CH2), allyl (—CH2CH═CH2), cyclopentenyl (—C₅H₇), and 5-hexenyl (—CH2CH2CH2CH2CH═CH2). The double bond may be in the cis or trans configuration.

The terms “C₂₋₁₈ alkynyl” and “C₃₋₁₀ cycloalkynyl” as used herein refer respectively C₂-C₁₈ normal, secondary, tertiary or the C₃₋₁₀ cyclic hydrocarbon with at least one site (usually 1 to 3, preferably 1) of unsaturation, i.e. a carbon-carbon, sp triple bond. Examples include, but are not limited to: acetylenic (—C≡CH) and propargyl (—CH2C≡CH).

The terms “C₁₋₁₈ alkylene” as used herein each refer to a saturated, branched or straight chain hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH2) 1,2-ethyl (—CH2CH2-), 1,3-propyl (—CH2CH2CH2-), 1,4-butyl (—CH2CH2CH2CH2-), and the like.

The terms “C₂₋₁₈ alkenylene” and “C₃₋₁₀ cycloalkenylene” as used herein refer to an unsaturated branched chain, straight chain, and respectively a cyclic hydrocarbon radical of 2-18 respectively 3-10 carbon atoms, and having two monovalent radical centers derived by the removal of two hydrogen atoms from the same or two different carbon atoms of a parent alkene, i.e. double carbon-carbon bond moiety. Typical alkenylene radicals include, but are not limited to: 1,2-ethylene (—CH═CH—).

The terms “C₂₋₁₈ alkynylene” and “C₃₋₁₀ cycloalkynylene” as used herein refer respectively to an unsaturated, branched or straight chain of 2-18 carbon atoms or to a cyclic hydrocarbon radical of 3-10 carbon atoms respectively, having two monovalent radical centers derived by the removal of two hydrogen atoms from the same or two different carbon atoms of a parent alkyne, i.e. triple carbon-carbon bond moiety. Typical alkynylene radicals include, but are not limited to: acetylene (—C≡C—), propargyl (—CH2C≡C—), and 4-pentynyl (—CH2CH2CH2C≡CH—).

The term “aryl” as used herein means a mono- or polycyclic aromatic hydrocarbon radical of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, spiro, anthracene, biphenyl, and the like.

“Arylalkyl” as used herein refers to an alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

The term “heterocyclic ring” as used herein means a mono- or polycyclic, saturated or mono-unsaturated or polyunsaturated monovalent hydrocarbon group having from 3 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl or selenocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, selenium and phosphorus, also including radicals wherein a heterocyclic ring is fused to one or more aromatic hydrocarbon rings for instance in the form of benzo-fused, dibenzo-fused and naphto-fused heterocyclic radicals; within this definition are included heterocyclic groups such as, but not limited to pyridyl, 2H-chromene, dihydroypyridyl, tetrahydropyridyl(piperidyl), thiazolyl, tetrahydrothiophenyl, sulfur oxidized tetrahydrothiophenyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, 3-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl.

Heteroaryl means pyridyl, dihydropyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl.

By way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrmidinyl, 5-pyrmnidinyl, 6-pyrmidinyl, 2-pyrazinyl, 3-pyrazinyl, 5-pyrazinyl, 6-pyrazinyl, 2-thiazolyl, 4-thiazolyl, or 5-thiazolyl.

By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or 3-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” means a saturated, unsaturated or aromatic ring system having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. Carbocycle thus includes some aryl groups.

As used herein and unless otherwise stated, the terms “C₁₋₁₈ alkoxy”, “C₃₋₁₀ cycloalkoxy”, “aryloxy”, “arylalkyloxy”, “oxyheterocyclic ring”, “thio C₁₋₇ alkyl”, thio C₃₋₁₀ cycloalkyl”, “arylthio”, “arylalkylthio” and “thioheterocyclic ring” refer to substituents wherein a C₁₋₁₈ alkyl radical, respectively a C₃₋₁₀ cycloalkyl, aryl, arylalkyl or heterocyclic ring radical (each of them such as defined herein), are attached to an oxygen atom or a sulfur atom through a single bond, such as but not limited to methoxy, ethoxy, propoxy, butoxy, thioethyl, thiomethyl, phenyloxy, benzyloxy, mercaptobenzyl and the like.

As used herein and unless otherwise stated, the term halogen means any atom selected from the group consisting of fluorine, chlorine, bromine and iodine.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

The compounds of the invention optionally are bound covalently to an insoluble matrix and used for affinity chromatography (separations, depending on the nature of the groups of the compounds, for example compounds with pendant aryl are useful in hydrophobic affinity separations.

It has been shown in the present invention that novel benzimidazole conjugates, with as an example the benzimidazole-coumarin conjugates show potent antiviral activity. The benzimidazole conjugates have been synthesised according to the following general procedures exemplified with some specific benzimidazole-coumarin conjugates. These procedures can however be applied by a person skilled in the art to other compounds of the invention.

For the synthesis of the new benzimidazoles, like 4a-k and the corresponding glucopyranosides, like 7a-h, various substituted phenylenediamines 1a-f were treated with carbon disulfide (Klinesová, V. et al. J. Eur. J. Med. Chem. 2002, 37, 409-418) and ethanolic KOH in H₂O (see Scheme 1). Then NH₄OH and 3-chloromethylcoumarins 3a-c were added in sequence to afford the desired benzimidazole-SCH₂-coumraines 4a-k. The overall yields were around 53-86%.

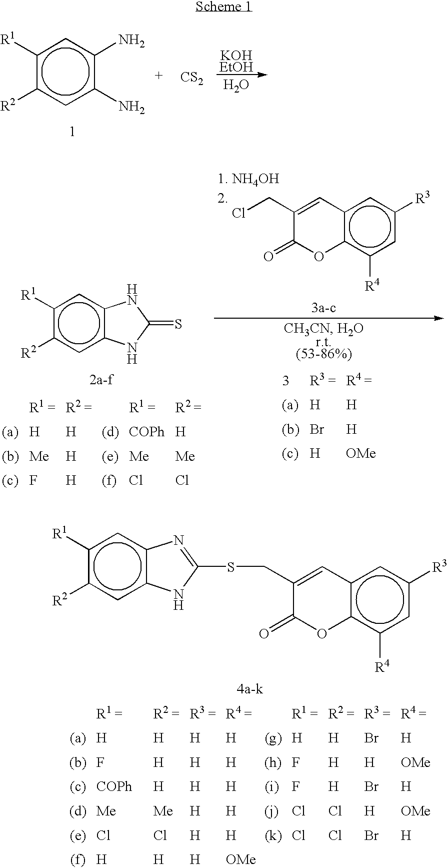

Direct coupling of conjugated heterocycles 4a-k with O-acetylpyranose 5 met with failure. Thus we silylated 2-thiones 2a-f with N,O-bistrimethylsilylactamide (BSA) (Bhan, P. et al. Nucleic Acids Res. 1997, 25, 3310-3317 and Khodair, A. I. et al. Nucleosides, Nusleotides & Nucleic Acids 2003, 22, 2061-2076) first; the intermediates were then coupled with O-peracetylpyranose 5 in presence of Me₃SiOTf at 80° C. (Scheme 2). Then the 2-thione intermediates 6a-e were alkylated with various 3-(chloromethyl)coumarins 3a-c in the presence of NH₄OH (Klimesová, V. et al. J. Eur. J. Med. Chem. 2002, 37, 409-418) at room temperature to give the desired of N-(glucopyranosyl)benzimidazole 7a-h. The obtained products can than be deprotected with procedures known to the person skilled in the art, such as for deacetylation by using ammonia (see scheme 3).

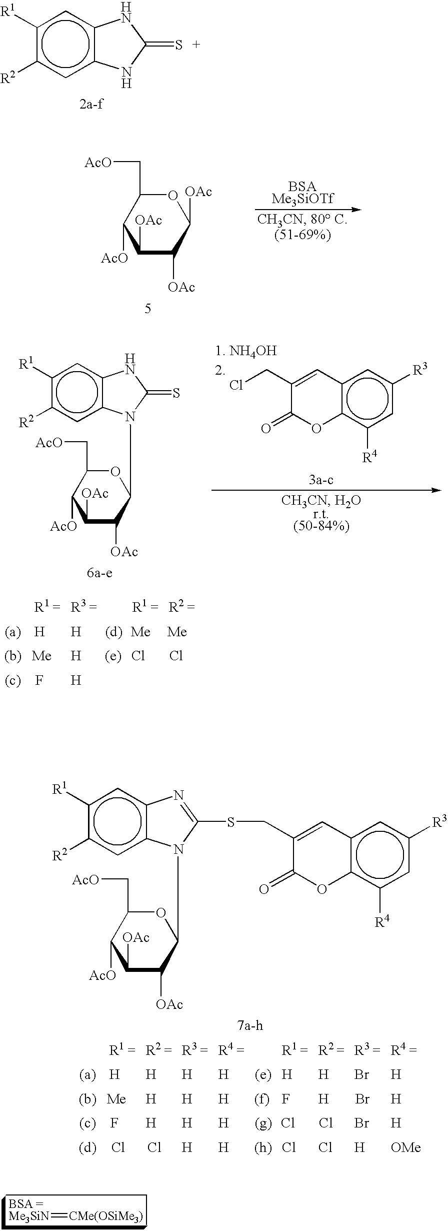

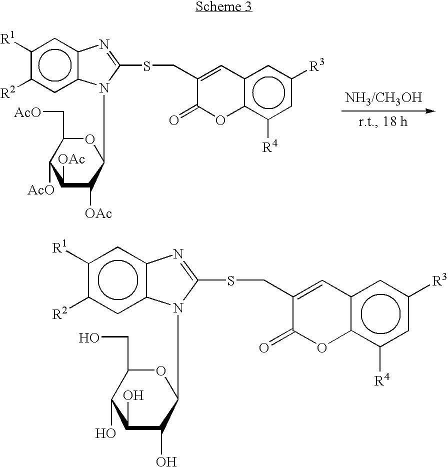

In some examples, the coumarine moiety was replaced with other heterocyclic rings or aryl rings. The synthesis was in general performed as shown in scheme 4. The thiones 2 were coupled with 2-(chloromethyl)naphthalene (8), benzyl chloride (10a), or 4-(chloromethyl)pyridine (10b) in the presence of NH₄OH in acetonitrile and water. Benzimidazole analogs 9 and 11a-d were produced in 51-77% yields.

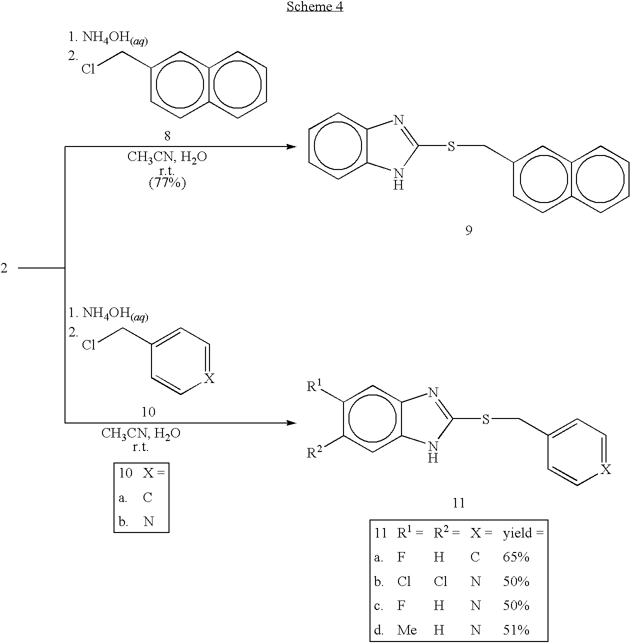

Furthermore, 2-mercaptobenzimidazole (2a) was silylated with N,O-bistrimethylsilylactamide (13SA); the intermediate was then coupled with O-peracetylpyranose 7 in the presence of Me₃SiOTf at 80° C. (Scheme 2). The resultant intermediates 8 were alkylated with p-nitrobenzyl bromide (9) at room temperature to give N-(glucopyranosyl)benzimidazole 10.

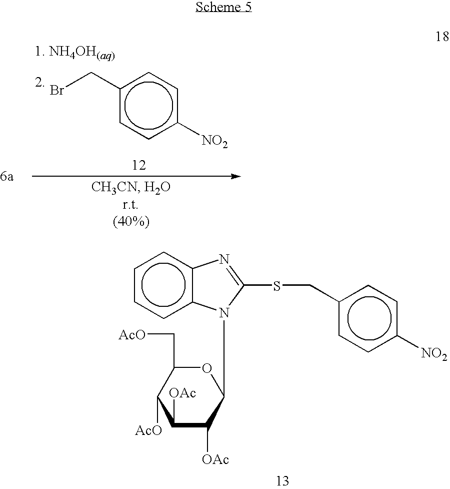

Also imidazopyridine and purine derivatives were synthesised. We followed the synthetic procedures shown in Scheme 6 to produce the intermediate 15a by using the commercially available heterocyclic compound 2,3-diaminopyridine (14a) as the starting material (see Scheme 1). Coupling of 15a with 3-(chloromethyl)coumarins 3 gave the desired imidazopyridine-coumarin conjugates 16a-c. Under the same conditions, we successfully led commercially available 4,5-diaminopyrimidine (14b) to the purine-coumarin conjugates 16d and 16e. The overall yields of 16a-e ranged from 40-83%.

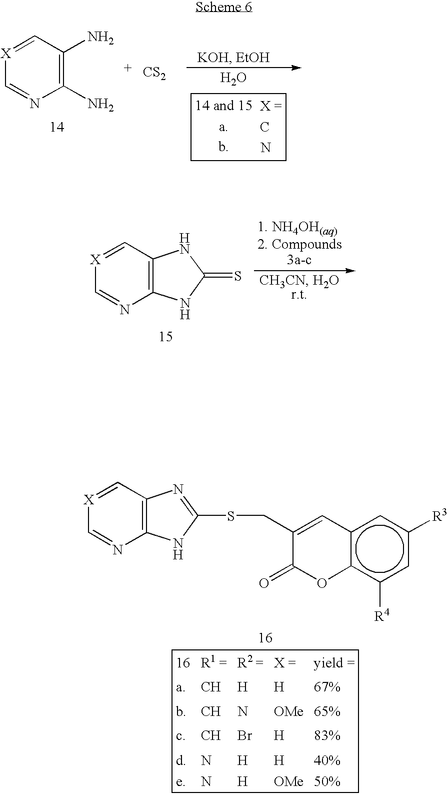

Furthermore, the benzoxazole and benzothiazole derivatives 19 were prepared in general according to scheme 7. We treated the starting materials 2-aminophenol (17a) and 2-aminobenzenethiol (17b), respectively, with carbon disulfide and 3-(chloromethyl)cumarins 3 in sequence. Five new compounds in the family of benzoxazole-coumarin conjugate (i.e., 19a-c) and benzothiazole-coumarin conjugate (i.e., 19d and 19e) were produced in good yields (69-89%).

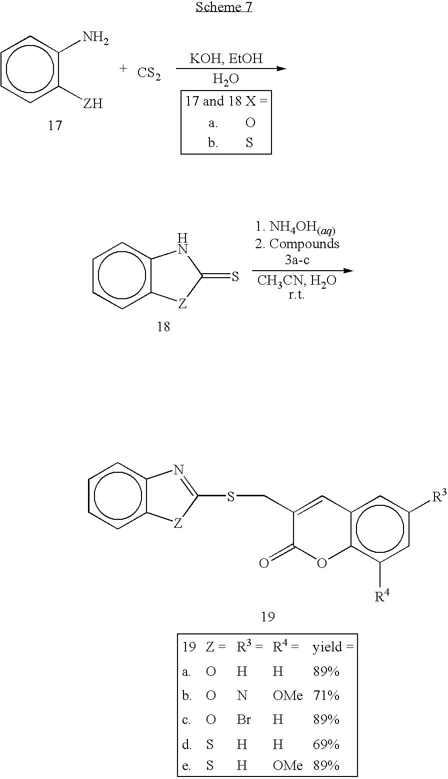

Finally, we attached a pyranose moiety onto 2-thiones 2a,b as shown in Scheme 8. Reaction of 2a,b with O-peracety-2-deoxy-β-D-glucose (20) in the presence of Me₃SiOTf generated intermediates 21a,b. The thione moiety therein allowed these compounds to couple with coumarins 3 to produce the desired targets 22a-c in 54-92% yields. Our identification of the structures is exemplified by use of 22b to stand for the target molecules. The mass spectrum of 22b in the positive ion mode under electrospray ionization method exhibited a peak at 611.30 for the species [M+H]⁺, which indicates the molecular formula to be C₃₀H₃₀N₂O₁₀S. The three moieties joined together included benzimidazole, coumarin, and dexoyglucopyranose. The —N═C(—N)(—S) carbon resonated at the 149.35 ppm in the ¹³C NMR spectrum. On the other hand, the glycosidic proton resonated at the 5.75 ppm and the two diastereotopic SCH₂ protons appeared at 4.58 and 4.49 ppm as two doublets with J=13.6 Hz in its ¹H NMR spectrum.

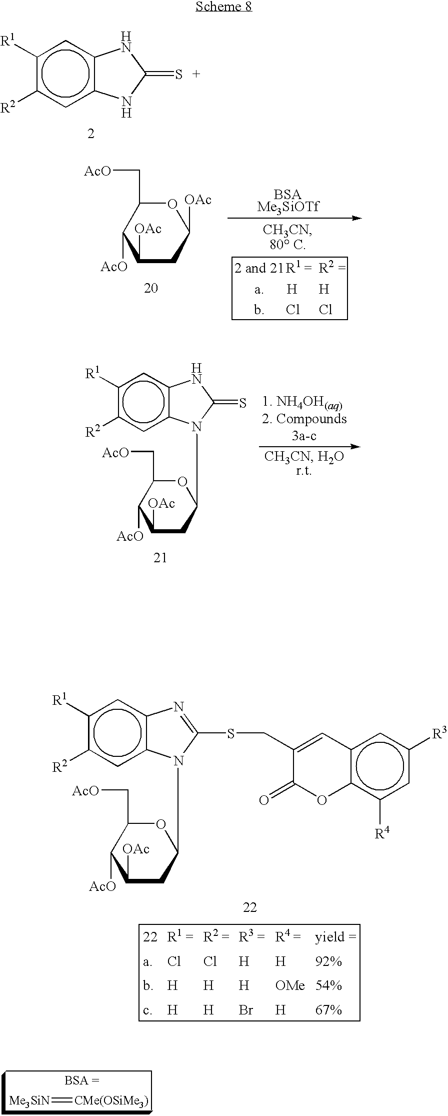

In antiviral assays for detection of anti-HCV activity, we found that the newly synthesized benzimidazole-coumarin conjugates were active towards HCV in the assays described herein. As an example, compound 4k and the corresponding glucoside 7g exhibited potent activity against HCV with EC₅₀ values of 2.29 μM and 4.08 μM, respectively and selectivity values were found high, which were 19.3 and >15.9, respectively. Some results are shown in Table 1, which indicate the 50% inhibitory concentrations for the inhibition of viral replication (EC₅₀) and host growth (CC₅₀), as well as the selectivity index (SI═CC₅₀/EC₅₀).

The compounds of the invention can be employed for the treatment or prophylaxis of viral infections, more particularly Flaviviral infections, in particular of HCV. When using one or more derivatives of the formulae (I), (II), (III), (IV), (VI), (VII) and (VIII) as defined herein:
the active ingredients of the compound(s) may be administered to the mammal (including a human) to be treated by any means well known in the art, i.e. orally, intranasally, subcutaneously, intramuscularly, intradermally, intravenously, intra-arterially, parenterally or by catheterization.
the therapeutically effective amount of the preparation of the compound(s), especially for the treatment of viral infections in humans and other mammals, preferably is a flaviviral or herpesviridae enzyme inhibiting amount. More preferably, it is a flaviviral replication inhibiting amount or a flaviviral enzyme inhibiting amount of the derivative(s) of formula (I) as defined herein corresponds to an amount which ensures a plasma level of between 1 μg/ml and 100 mg/ml, optionally of 10 mg/ml. Depending upon the pathologic condition to be treated and the patient's condition, the said effective amount may be divided into several sub-units per day or may be administered at more than one day intervals.

The present invention further relates to a method for preventing or treating a viral infections in a subject or patient by administering to the patient in need thereof a therapeutically effective amount of compounds of the present invention. The therapeutically effective amount of the preparation of the compound(s), especially for the treatment of viral infections in humans and other mammals, preferably is a flaviviral or herpesviridae enzyme inhibiting amount. More preferably, it is a flaviviral or herpesviridae replication inhibiting amount or a flaviviral or herpesviridae enzyme inhibiting amount of the derivative(s) of formula (I), (II), (III), (IV), (VI), (VII) or (VIII) as defined herein. Depending upon the pathologic condition to be treated and the patient's condition, the said effective amount may be divided into several sub-units per day or may be administered at more than one day intervals.

The present invention also relates to a combination of different antiviral drugs of the invention or to a combination of the antiviral drugs of the invention with other drugs that exhibit anti-HCV activity.

The invention also relates to a pharmaceutical composition or combined preparation of antiviral drugs and containing:

### Either:

### A)

(a) a combination of two or more of the compounds of the present invention, and
(b) optionally one or more pharmaceutical excipients or pharmaceutically acceptable carriers, for simultaneous, separate or sequential use in the treatment or prevention of a viral infection or

### B)

(c) one or more anti-viral agents, and
(d) at least one of the compounds of the present invention, and
(e) optionally one or more pharmaceutical excipients or pharmaceutically acceptable carriers, for simultaneous, separate or sequential use in the treatment or prevention of a viral infection.

Suitable anti-viral agents for inclusion into the antiviral compositions or combined preparations of this invention include, for instance, interferon-alfa (either pegylated or not), ribavirin and other selective inhibitors of the replication of HCV, such as a compound falling within the scope of disclosure EP1162196, WO 03/010141, WO 03/007945 and WO 03/010140, a compound falling within the scope of disclosure WO 00/204425, and other patents or patent applications within their patent families or all the foregoing filings and/or an inhibitor of flaviviral protease and/or one or more additional flavivirus polymerase inhibitors.

The pharmaceutical composition or combined preparation with activity against viral infection according to this invention may contain the compounds of the present invention over a broad content range depending on the contemplated use and the expected effect of the preparation. Generally, the content of the benzimidazole conjugates derivatives of the present invention of the combined preparation is within the range of 0.1 to 99.9% by weight, preferably from 1 to 99% by weight, more preferably from 5 to 95% by weight.

When using a pharmaceutical composition of combined preparation:
the active ingredients may be administered to the mammal (including a human) to be treated by any means well known in the art, i.e. orally, intranasally, subcutaneously, intramuscularly, intradermally, intravenously, intra-arterially, parenterally or by catheterization.
the therapeutically effective amount of each of the active agents, especially for the treatment of viral infections in humans and other mammals, particularly is a flaviviral enzyme inhibiting amount.

When applying a combined preparation, the active ingredients may be administered simultaneously but it is also beneficial to administer them separately or sequentially, for instance within a relatively short period of time (e.g. within about 24 hours) in order to achieve their functional fusion in the body to be treated.

The invention also relates to the compounds of formula (I), (II), (III), (IV), (VI), (VII) and (VII) being used for inhibition of the proliferation of other viruses than HCV, particularly for the inhibition of other members of the family of the Flaviviridae, including but not limited to the Yellow fever virus, the Dengue fever virus, West Nile virus, Japanese encephalitis virus, hepatitis G virus, bovine viral diarrhea virus, classical swine fever virus, border disease virus but also for the inhibition of other viruses including HIV and other retroviruses.

The present invention further provides veterinary compositions comprising at least one active ingredient as above defined together with a veterinary carrier therefor, for example in the treatment of BVDV. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

More generally, the invention relates to the compounds of formula (I), (II), (III), (IV), (VI), (VII) and (VIII) being useful as agents having biological activity (particularly antiviral activity) or as diagnostic agents. Any of the uses mentioned with respect to the present invention may be restricted to a non-medical use, a non-therapeutic use, a non-diagnostic use, or exclusively an in vitro use, or a use related to cells remote from an animal.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds of formula (I) are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formula (I) as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formula (I) may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formula (I) may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCeI™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of formula (I) may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or allkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one

C8C₂₋₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw”, 2 d ed. (Hanser Verlag, Vienna, 1981) and “Encyclopaedia of Surfactants, (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene, glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical adiministration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, imiediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

The compounds of formula (I), can be prepared while using a series of chemical reactions well known to those skilled in the art, altogether making up the process for preparing said compounds and exemplified further. The processes described further are only meant as examples and by no means are meant to limit the scope of the present invention.

### EXAMPLES

The following examples illustrate the present invention without being limited thereto. Part A represent the preparation of the benzimidazole conjugates whereas Part B represents the pharmacological examples. Analoguous compounds of the ones specifically mentioned herein are synthesised in the same fashion as in the foregoing schemes and following examples by varying the starting materials, intermediates, solvents and conditions as will be known by those skilled in the art.

TABLE 1
Examples of the compounds of the invention: benzimidazole-coumarine conjugates

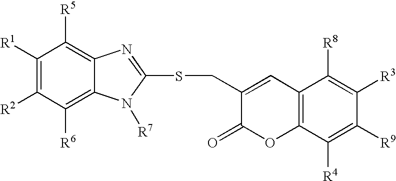

| Compound | | | | | | | | | | |
|---|---|---|---|---|---|---|---|---|---|---|
| Code | R⁵ | R¹ | R² | R⁶ | R⁷ | R⁸ | R³ | R⁹ | R⁴ | Name |
| 4a | H | H | H | H | H | H | H | H | H | 3-(1H-Benzoimidazol-2-ylsulfanylmethyl)-chromen- |
| | | | | | | | | | | 2-one |
| 4b | H | F | H | H | H | H | H | H | H | 3-(5-Fluoro-1H-benzoimidazol-2-ylsulfanylmethyl)- |
| | | | | | | | | | | chromen-2-one |
| 4c | H | CO- | H | H | H | H | H | H | H | 3-(5-Benzoyl-1H-benzoimidazol-2- |
| | | phenyl | | | | | | | | ylsulfanylmethyl)-chromen-2-one |
| 4d | H | Me | Me | H | H | H | H | H | H | 3-(5,6-Dimethyl-1H-benzoimidazol-2- |
| | | | | | | | | | | ylsulfanylmethyl)-chromen-2-one |
| 4e | H | Cl | Cl | H | H | H | H | H | H | 3-(5,6-Dichloro-1H-benzoimidazol-2- |
| | | | | | | | | | | ylsulfanylmethyl)-chromen-2-one |
| 4f | H | H | H | H | H | H | H | H | Me | 3-(1H-benzoimidazol-2-ylsulfanylmethyl)-8-methyl- |
| | | | | | | | | | | chromen-2-one |
| 4g | H | H | H | H | H | H | Br | H | H | 3-(1H-Benzoimidazol-2-ylsulfanylmethyl)-6-bromo- |
| | | | | | | | | | | chromen-2-one |
| 4h | H | F | H | H | H | H | H | H | OMe | 3-(5-Fluoro-1H-benzoimidazol-2-ylsulfanylmethyl)- |
| | | | | | | | | | | 8-methoxy-chromen-2-one |
| 4i | H | F | H | H | H | H | Br | H | H | 6-Bromo-3-(5-fluoro-1H-benzoimidazol-2- |
| | | | | | | | | | | ylsulfanylmethyl)-chromen-2-one |
| 4j | H | Cl | Cl | H | H | H | H | H | OMe | 3-(5,6-Dichloro-1H-benzoimidazol-2- |
| | | | | | | | | | | ylsulfanylmethyl)-8-methoxy-chromen-2-one |
| 4k | H | Cl | Cl | H | H | H | Br | H | H | 6-Bromo-3-(5,6-dichloro-1H-benzoimidazol-2- |
| | | | | | | | | | | ylsulfanylmethyl)-chromen-2-one |
| 7a | H | H | H | H | 2′,3′,4′,6′-Tetra- | H | H | H | H | Acetic acid 3,5-diacetoxy-2-acetoxymethyl-6-[2-(2- |
| | | | | | O- | | | | | oxo-2H-chromen-3-ylmethylsulfanyl)- |
| | | | | | acetylglucopyranosyl | | | | | benzoimidazol-1-yl]-tetrahydro-pyran-4-yl ester |
| 7b | H | Me | H | H | 2′,3′,4′,6′-Tetra- | H | H | H | H | Acetic acid 3,5-diacetoxy-2-acetoxymethyl-6-[5- |
| | | | | | O- | | | | | methyl-2-(2-oxo-2H-chromen-3-ylmethylsulfanyl)- |
| | | | | | acetylglucopyranosyl | | | | | benzoimidazol-1-yl]-tetrahydro-pyran-4-yl ester |
| 7c | H | F | H | H | 2′,3′,4′,6′-Tetra- | H | H | H | H | Acetic acid 3,5-diacetoxy-2-acetoxymethyl-6-[5- |
| | | | | | O- | | | | | fluoro-2-(2-oxo-2H-chromen-3-ylmethylsulfanyl)- |
| | | | | | acetylglucopyranosyl | | | | | benzoimidazol-1-yl]-tetrahydro-pyran-4-yl ester |
| 7d | H | Cl | Cl | H | 2′,3′,4′,6′-Tetra- | H | H | H | H | Acetic acid 3,5-diacetoxy-2-acetoxymethyl-6-[5,6- |
| | | | | | O- | | | | | dichloro-2-(2-oxo-2H-chromen-3-ylmethylsulfanyl)- |
| | | | | | acetylglucopyranosyl | | | | | benzoimidazol-1-yl]-tetrahydro-pyran-4-yl ester |
| 7e | H | H | H | H | 2′,3′,4′,6′-Tetra- | H | Br | H | H | Acetic acid 4,5-diacetoxy-2-acetoxymethyl-6-[2-(6- |
| | | | | | O- | | | | | bromo-2-oxo-2H-chromen-3-ylmethylsulfanyl)- |
| | | | | | acetylglucopyranosyl | | | | | benzoimidazol-1-yl]-tetrahydro-pyran-3-yl ester |
| 7f | H | F | H | H | 2′,3′,4′,6′-Tetra- | H | Br | H | H | Acetic acid 3,5-diacetoxy-2-acetoxymethyl-6-[2-(6- |
| | | | | | O- | | | | | bromo-2-oxo-2H-chromen-3-ylmethylsulfanyl)-5- |
| | | | | | acetylglucopyranosyl | | | | | fluoro-benzoimidazol-1-yl]-tetrahydro-pyran-4-yl |
| | | | | | | | | | | ester |
| 7g | H | Cl | Cl | H | 2′,3′,4′,6′-Tetra- | H | Br | H | H | Acetic acid 3,5-diacetoxy-2-acctoxymethyl-6-[2-(6- |
| | | | | | O- | | | | | bromo-2-oxo-2H-chromen-3-ylmethylsulfanyl)-5,6- |
| | | | | | acetylglucopyranosyl | | | | | dichloro-benzoimidazol-1-yl]-tetrahydro-pyran-4-yl |
| | | | | | | | | | | ester |
| 7h | H | Cl | Cl | H | 2′,3′,4′,6′-Tetra- | H | H | H | OMe | Acetic acid 3,4,5-triacetoxy-6-[5,6-dichloro-2-(8- |
| | | | | | O- | | | | | methoxy-2-oxo-2H-chromen-3-ylmethylsulfanyl)- |
| | | | | | acetylglucopyranosyl | | | | | benzoimidazol-1-yl]-tetrahydro-pyran-2-ylmethyl |
| | | | | | | | | | | ester |
| 22a | H | Cl | Cl | H | 3′,4′,6′-Tri-O- | H | H | H | H | Acetic acid 3,4-diacetoxy-6-[5,6-dichloro-2-(2-oxo- |
| | | | | | acetylglucopyranosyl | | | | | 2H-chromen-3-ylmethylsulfanyl)-benzoimidazol-1- |
| | | | | | | | | | | yl]-tetrahydro-pyran-2-ylmethyl ester |
| 22b | H | H | H | H | 3′,4′,6′-Tri-O- | H | H | H | OMe | Acetic acid 3,4-diacetoxy-6-[2-(8-methoxy-2-oxo- |
| | | | | | acetylglucopyranosyl | | | | | 2H-chromen-3-ylmethylsulfanyl)-benzoimidazol-1- |
| | | | | | | | | | | yl]-tetrahydro-pyran-2-ylmethyl ester |
| 22c | H | H | H | H | 3′,4′,6′-Tri-O- | H | Br | H | H | Acetic acid 3,4-diacetoxy-6-[2-(6-bromo-2-oxo-2H- |
| | | | | | acetylglucopyranosyl | | | | | chromen-3-ylmethylsulfanyl)-benzoimidazol-1-yl]- |
| | | | | | | | | | | tetrahydro-pyran-2-ylmethyl ester |

TABLE 2
Examples of the compounds of the invention: pyridine-imidazole-coumarine
conjugates, purine-coumarine conjugates, benzimidazole-naphtyk conjugates, benzimidazole-
pyridine conjugates, benzooxazole-coumarine conjugates and benzothiazole-coumarine
conjugates

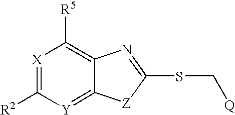

| Compound | | | | | | | |
|---|---|---|---|---|---|---|---|
| Code | R⁵ | X | R² | Y | Z | Q | Name |
| 9 | H | CH | H | CH | NH | 2-naphthyl | 2-(Naphthalen-2-ylmethylsulfanyl)-1H- |
| | | | | | | | benzoimidazole |
| 11a | H | C—F | H | CH | NH | Phenyl | 2-Benzylsulfanyl-5-fluoro-1H- |
| | | | | | | | benzoimidazole |
| 11b | H | C—Cl | Cl | CH | NH | 4-pyridyl | 5,6-Dichloro-2-(pyridin-4-ylmethylsulfanyl)- |
| | | | | | | | 1H-benzoimidazole |
| 11c | H | C—F | H | CH | NH | 4-pyridyl | 5-fluoro-2-(pyridin-4-ylmethylsulfanyl)-1H- |
| | | | | | | | benzoimidazole |
| 11d | H | C—Me | H | CH | NH | 4-pyridyl | 5-methyl-2-(pyridin-4-ylmethylsulfanyl)-1H- |
| | | | | | | | benzoimidazole |
| 13 | H | CH | H | CH | N-2′,3′,4′,6′- | 4-nitro-phenyl | Acetic acid 3,4,5-triacetoxy-6-[2-(4-nitro- |
| | | | | | Tetra-O- | | benzylsulfanyl)-benzoimidazol-1-yl]- |
| | | | | | acetylglucopyranosyl | | tetrahydro-pyran-2-yl |
| | | | | | | | methyl ester |
| 16a | H | CH | H | N | NH | chromen-2-on-3-yl | 3-(3H-Imidazo[4,5-b]pyridin-2- |
| | | | | | | | ylsulfanylmethyl)-chromen-2-one |
| 16b | H | CH | H | N | NH | 8-methoxy- | 3-(3H-Imidazo[4,5-b]pyridin-2- |
| | | | | | | chromen-2-on-3-yl | ylsulfanylmethyl)-8-methoxy-chromen-2-one |
| 16c | H | CH | H | N | NH | 6-bromo-chromen- | 3-(3H-Imidazo[4,5-b]pyridin-2- |
| | | | | | | 2-on-3-yl | ylsulfanylmethyl)-6-bromo-chromen-2-one |
| 16d | H | N | Cl | N | NH | chromen-2-on-3-yl | 3-(2-Chloro-9H-purin-8-ylsulfanylmethyl)- |
| | | | | | | | chromen-2-one |
| 16e | H | N | Cl | N | NH | 8-methoxy- | 3-(2-Chloro-9H-purin-8-ylsulfanylmethyl)-8- |
| | | | | | | chromen-2-on-3-yl | methoxy-chromen-2-one |
| 19a | H | CH | H | CH | O | chromen-2-on-3-yl | 3-(Benzooxazol-2-ylsulfanylmethyl)- |
| | | | | | | | chromen-2-one |
| 19b | H | CH | H | CH | O | 8-methoxy- | 3-(Benzooxazol-2-ylsulfanylmethyl)-8- |
| | | | | | | chromen-2-on-3-yl | methoxy-chromen-2-one |
| 19c | H | CH | H | CH | O | 6-bromo-chromen- | 3-(Benzooxazol-2-ylsulfanylmethyl)-6- |
| | | | | | | 2-on-3-yl | bromo-chromen-2-one |
| 19d | H | CH | H | CH | S | chromen-2-on-3-yl | 3-(Benzothiazol-2-ylsulfanylmethyl)- |
| | | | | | | | chromen-2-one |
| 19e | H | CH | H | CH | S | 8-methoxy- | 3-(Benzothiazol-2-ylsulfanylmethyl)-8- |
| | | | | | | chromen-2-on-3-yl | methoxy-chromen-2-one |

### Part A: Preparation of the Compounds of the Invention

### General Procedures Used for the Preparation of all the Exemplified Compounds as Described Herein.

All reactions were carried out in oven-dried glassware (110° C.) under an atmosphere of nitrogen unless as indicated otherwise. Chloroform, ethanol, ethylacetate (EtOAc), and hexanes were purchased from Mallinckrodt Chemical Co. Hexanes were dried and distilled from CaH₂. 3,4-Diaminobenzoic acid, 1,2-diamino-4,5-dichlorobenzene, 1,2-diamino-4,5-dimethylbenzene, 1,2-diamino-4-fluorobenzene, 3,4-diaminotoluene, and o-phenelenediamine were purchased from Aldrich Chemical Co. D-(+)-Glucose was purchased from Sigma Chemical Co. Salicyldehyde and trimethylsilyl trifluoromethanesulfonate (TMSOTf) were purchased from Fluka Chemika. N,O-Bistrimethylsilylacetamide (BSA) was purchased from Acros Chemical Co. Acetonitrile was purchased from Fischer Scientific Co. Carbodisulfide was purchased from Showa Chemical Co. Acetic acid, acetic anhydide, and hydrochloric acid were purchased from Riedel-de Haen Chemical Co. Potassium hydroxide was purchased from Merck Inc. Benzimidazole-2-thiones, 3-chloromethylcoumarins, and 1′,2′,3′,4′,6′-penta-O-acetylglucose were prepared according to the literature methods.

Analytical thin layer chromatography (TLC) was performed on precoated plates (silica gel 60 F-254), purchased from Merck Inc. Purification by gravity column chromatography was carried out by use of Merck Reagents Silica Gel 60 (particle size 0.063-0.200 mm, 70-230 mesh ASTM). Infrared (1R) spectra were measured on a Bomem Michelson Spectrometer FT-IR. Absorption intensities are recorded by the following abbreviations: s, strong; m, medium; w, weak; br, broad. High-resolution mass spectra were obtained by means of a JEOL JMS-HX110 mass Spectrometer. Proton NMR spectra were obtained on a Varian Mercury-400 (400 MHz) spectrometer by use of chloroform-d, dimethylsulfoxide-d₆ as solvents and tetramethylsilane as an internal standard. Carbon-13 NMR spectra were performed on a Varian Mercury-400 (100 MHz) spectrometer by use of chloroform-d and dimethylsulfoxide-d₆ as solvents. Carbon-13 chemical shifts are referenced to the center of the CDCl₃ triplet (□177.0 ppm) and DMSO-d₆ pentet (□39.54 ppm). Multiplicities are recorded by the following abbreviations: s, singlet; d, doublet; t, triplet; q, quartet; m, multiplet; J, coupling constant (Hz).

### Example 1

### Standard Procedure A for the Preparation of Coumarinyl(methylthio)benzimidazoles (4a-k)

To a solution containing benzimidazole-2-thiones 2 (1.0 equiv) in acetonitrile (15 mL) was added aqueous ammonium hydroxide (12 drops). After the solution was stirred at room temperature for 30 min, 3-(chloromethyl)coumarins 3 (1.0 equiv) in acetonitrile (2.0 mL) was added to the mixture. The mixture was stirred for another 18 h to give precipitates. The crude solids were collected by vacuum filtration. The residues were purified by use of column chromatography packed with silica gel and eluented with a mixture of EtOAc and hexanes.

The resultant solids were recrystallizd with a mixture of EtOAc and hexanes to give coumarinyl(methylthio)benzimidazoles 4a-k with purity >99.5%, as checked by GC.

### Example 2

### Preparation of 2-(6′-Bromocoumarinyl)methylthio-5-fluorobenzimidazole (4i)

The Standard Procedure A was followed by use of 5-fluorobenzimidazole-2-thione (2c, 84.1 mg, 0.499 mmol, 1.0 equiv) and 3-chloromethyl-6-bromocoumarin (3c, 137.1 mg, 0.4999 mmol, 1.0 equiv). After workup and purification, the solids were recrystallizd with 40% EtOAc in hexanes to give 4i (174.1 mg, 0.4296 mmol) in 86% yield as white needles: mp (recrystallized from EtOAc) 123.0-124.3° C.; ¹H NMR (DMSO-d₆, 400 MHz) δ 7.95 (s, 1H, ArH), 7.61 (d, J=1.6 Hz, 1H, ArH), 7.52 (dd, J=7.2, 2.4 Hz, 1H, ArH), 7.30-7.33 (m, 1H, ArH), 7.15 (d, J=7.2 Hz, 1H, ArH), 7.08 (dd, J=7.6, 2.0 Hz, 1H, ArH), 6.81 (m, 1H, ArH), 4.33 (s, 2HSCH2); ¹³C NMR (DMSO-d₆, 100 MHz) □159.60, 159.21, 157.32, 151.68, 149.94, 139.33, 133.57, 139.84, 125.55, 120.28, 117.68, 116.27, 113.94, 109.21, 109.00, 100.07, 30.66; IR (KBr) 3224 (br), 1703 (s), 1627 (m), 1562 (w), 1436 (m), 1395 (m), 1191 (m), 1087 (m), 955 (w), 817 (m), 634 (w) cm⁻¹; HRMS m/z calcd for C₁₇H₁₀BrFN₂O₂S: 403.9630, found 403.9631.

### Example 3

### Standard Procedure B for the Preparation of 1-(tetra-O-Acetyl)glucopyranosyl-benzimidazole-2-thiones (6a-e) and Deacetylated Analogs Thereof

To a solution containing benzimidazole-2-thiones 2 (1.0 equiv) in dry acetonitrile (40 mL) was added BSA (1.5 equiv) in nitrogen atmosphere. After the solution was stirred at room temperature for 15 min, 1′,2′,3′,4′,6′-penta-O-acetylglucopyranose (5, 1.0 equiv) in dry acetonitrile (2.0 mL) and TMSOTf (1.5 equiv) were added in sequence. The reaction mixture was heated to 80° C. and stirred for 18 h. Excess solvent was removed under reduced pressure and the residue was treated with 20% aqueous NaHCO₃ solution (10 mL). The aqueous phase was extracted with ethyl acetate (3×10 mL). The combined organic layers were washed with brine (5.0 mL), dried over MgSO₄ (s), and concentrated under reduced pressure to afford a residue. The residue was purified by use of column chromatography packed with silica gel and eluented with a mixture of EtOAc and hexanes to give the desired 6a-e with purity >99.5%, as checked by GC.

The acetyl protecting groups can be removed by using NH₃ in methanol at room temperature for 18 h. The acetyl protecting groups can be removed by using NH₃ in methanol at room temperature for 18 h.

### Example 4

### Preparation of 1-(2′,3′,4′,6′-Tetra-O-acetylglucopyranosyl)benzimidazole-2-thione (6a)

The Standard Procedure B was followed by use of benzimidazole-2-thione (2a, 1.71 g, 11.33 mmol, 1.0 equiv), BSA (3.456 g, 16.99 mmol, 1.5 equiv), glucose acetate 5 (4.42 g, 11.33 mmol, 1.0 equiv), and TMSOTf (3.77 g, 16.99 mmol, 1.5 equiv). After the reaction mixture was worked up, the residue was purified by use of column chromatography (80% EtOAc in hexanes as eluent) to give 6a (2.81 g, 5.84 mmol) in 52% yield as white solid foam: mp (recrystallized from EtOAc) 109.4-110.5° C.; ¹H NMR (CDCl₃, 400 MUfz) δ 10.68 (bs, 1H, NH), 7.56 (m, 1H, ArH), 7.23-7.31 (m, 3H, ArH), 6.55 (d, J=7.2 Hz, 1H, H1′), 5.68 (t, J=7.2 Hz, 1H, H2′), 5.59 (t, J=7.2 Hz, 1H, H3′), 5.39 (t, J=8.0 Hz, 1H, H4′), 4.28-4.37 (m, 2H, H6′), 4.09-4.12 (m, 1H, H5′), 2.19 (s, 3H, CH₃), 2.17 (s, 3H, CH₃), 2.07 (s, 3H, CH₃), 1.91 (s, 3H, CH₃); ¹³C NMR(CDCl₃, 100 MHz) δ 170.47, 169.77, 169.58, 169.32, 168.86, 130.40, 130.15, 124.04, 123.28, 111.59, 110.11, 83.17, 74.60, 72.77, 68.99, 67.84, 61.57, 20.66, 20.55, 20.50, 20.38; IR (KBr) 2957 (br), 1753 (s), 1455 (m), 1368 (m), 1224 (s), 1037 (s), 908 (m), 746 (m), 610 (m) cm⁻¹; HRMS m/z calcd for C₂₁H₂₄N₂O₉S: 480.1202, found 480.1203.

### Example 5

### Standard Procedure C for the Preparation of 1-(2′,3′,4′,6′-Tetra-O-acetyl)glucopyranosyl-2-coumarinyl(methylthio)benzimidazoles (7a-h) and the Deacetylated Analogs Thereof

To a solution containing (glucopyranosyl)thiones 6 (1.0 equiv) in acetonitrile (15 mL) was added aqueous solution of ammonium hydroxide (12 drops). After the solution was stirred at room temperature for 30 min, 3-(chloromethyl)coumarins 3 (1.0 equiv) in acetonitrile (2.0 mL) was added. The mixture was stirred for another 18 h to form precipitates. The crude solids were collected by vacuum filtration. The residues were purified by use of column chromatography packed with silica gel and eluented with a mixture of EtOAc and hexanes.

The resultant solids were recrystallizd with a mixture of EtOAc and hexanes to give (glucopyranosyl)benzimidazoles 7a-h with purity >99.5%, as checked by GC. The acetyl protecting groups can be removed by using NH₃ in methanol at room temperature for 18 h. The corresponding free polyols were obtained in 70-80% yields.

### Example 6

### Preparation of 1-(2′,3′,4′,6′-Tetra-O-acetyl glucopyranosyl-2-(6-bromocoumarinyl)-methythio-5,6-dichlorobenzimidazole (7g)

The Standard Procedure C was followed by use of 6e (54.9 mg, 0.099 mmol, 1.0 equiv) and 3-chloromethyl-6-bromocoumarin (3c, 27.4 mg, 0.099 mmol, 1.0 equiv). After workup and purification, the solids were recrystallizd with 30% EtOAc in hexanes to give 7g (66.1 mg, 0.084 mmol) in 84% yield as white needles: mp (recrystallized from EtOAc) 180.1-180.9° C.; ¹H NMR (CDCl₃, 400 MHz) δ 8.14 (s, 1H, ArH), 7.84 (s, 1H, ArH), 7.60-7.68 (m, 3H, 3×ArH), 7.18 (d, J=8.4 Hz, 1H, ArH), 5.55 (d, 1H, J=8.4 Hz, H1′), 5.36-5.39 (m, 3H, 12′, H3′, H4′), 4.84 (d, J=11 Hz, 1H, SCH), 4.40 (d, J=11 Hz, 1H, SCH), 4.22 (s, 2H, H6′), 3.98 (d, J=6.8 Hz, 1H, H5′), 2.15 (s, 3H, CH₃), 2.13 (s, 3H, CH₃), 2.06 (s, 3H, CH₃), 2.05 (s, 3H, CH₃); ¹³C NMR (CDCl₃, 100 MHz) δ 170.43, 169.82, 169.26, 168.48, 160.42, 152.36, 152.10, 141.15, 140.12, 134.65, 132.13, 130.57, 128.42, 127.85, 124.70, 120.40, 119.04, 118.20, 117.30, 113.76, 83.39, 74.80, 72.23, 69.32, 67.00, 60.72, 32.34, 20.67, 20.52, 20.48, 20.12; IR (KBr) 1746 (s), 1726 (s), 1595 (w), 1439 (m), 1367 (m), 1321 (w), 1225 (s), 1070 (m), 926 (w), 818 (w), 784 (w) cm⁻¹; HRMS m/z calcd for C₃₁H₂₇BrCl₂N₂O₁₁S: 783.9899, found 783.9896.

The compounds shown in Table 1 and 2 and the Schemes herein were prepared as described for the above examples.

### Part B: Methodology for and Results of the Determination of Antiviral and Cytostatic Activity

### Example 7

### Determination and Investigation of the Anti-HCV Activity

### Cells and Viruses

Madin-Darbey Bovine Kidney (MDBK) cells were maintained in Dulbecco's modified Eagle medium (DMEM) supplemented with BVDV-free 5% fetal calf serum (DMEME-FCS) at 37° C. in a humidified, 5% CO₂ atmosphere. BVDV-1 (strain PE515) was used to assess the antiviral activity in MDBK cells. Vero cells were maintained in the same way as MDBK cells. Vero cells were infected with Coxsackie B3 virus (strain Nancy).

### Determination of Cytostatic Effect on MDBK Cells

The effect of the drugs on exponentially growing MDBK cells was assessed as follows. Cells were seeded at a density of 5000 cell/well in 96 well plates in MEM medium (Gibco) supplemented with 10% fetal calf serum, 2 mM L-glutamine (Life Technologies) and bicarbonate (Life Technologies). Cells were cultured for 24 hr after which serial dilutions of the test compounds were added. Cultures were then again further incubated for 3 days after which the effect on cell growth was quantified by means of the MTS method (Promega). The concentration that results in 50% inhibition of cell growth is defined as the 50% cytostatic concentration (CC₅₀)

### Anti-HCV Assay/Replicon Assay

Huh-5-2 cells [a cell line with a persistent HCV replicon I3891uc-ubi-neo/NS3-3′/5.1; replicon with firefly luciferase-ubiquitin-neomycin phosphotransferase fusion protein and EMCV-IRES driven NS3-5B HCV polyprotein] was cultured in RPMI medium (Gibco) supplemented with 10% fetal calf serum, 2 mM L-glutamine (Life Technologies), 1× non-essential amino acids (Life Technologies); 100 IU/ml penicillin and 100 ug/ml streptomycin and 250 ug/ml G418 (Geneticin, Life Technologies). Cells were seeded at a densitiy of 7000 cells per well in 96 well View Plate™ (Packard) in medium containing the same components as described above, except for G418. Cells were allowed to adhere and proliferate for 24 hr. At that time, culture medium was removed and serial dilutions of the test compounds were added in culture medium lacking G418. Interferon alfa 2a (500 IU) was included as a positive control. Plates were further incubated at 37° C. and 5% CO₂ for 72 hours. Replication of the HCV replicon in Huh-5 cells results in luciferase activity in the cells. Luciferase activity is measured by adding 50 μl of 1× Glo-lysis buffer (Promega) for 15 minutes followed by 50 ul of the Steady-Glo Luciferase assay reagent (Promega). Luciferase activity is measured with a luminometer and the signal in each individual well is expressed as a percentage of the untreated cultures. Parallel cultures of Huh-5-2 cells, seeded at a density of 7000 cells/well of classical 96-well cell culture plates (Becton-Dickinson) are treated in a similar fashion except that no Glo-lysis buffer or Steady-Glo Luciferase reagent is added. Instead the density of the culture is measured by means of the MTS method (Promega).

### Quantitative Analysis of HCV RNA by Taqman Real-Time RT-PCR

Replicon cells were plated at 7.5×10³ cells per well in a 96-well plate plates at 37° C. and 5% CO₂ in Dulbecco's modified essential medium containing 10% fetal calf serum, 1% nonessential amino acids and 1 mg/ml Geneticin. After allowing 24 h for cell attachment, different dilutions of compound were added to the cultures. Plates were incubated for 5 days, at which time RNA was extracted using the Qiamp Rneazyi Kit (Qiagen, Hilden, Germany). A 50 μL PCR reaction contained TaqMan EZ buffer (50 mmol/L Bicine, 115 mmol/L potassium acetate, 0.01 mmol/L EDTA, 60 nmol/L 6-carboxy-X-rhodamine, and 8% glycerol, pH 8.2; Perkin Ehner Corp./Applied Biosystems), 300 μmol/L deoxyadenosine triphosphate, 300 μmol/L deoxyguanosine triphosphate, 300 μmol/L deoxycytidine triphosphate, 600 μmol/L deoxyuridine triphosphate, 200 μmol/L forward primer [5′-ccg gcT Acc Tgc ccA TTc], 200 tμmol/L reverse primer [ccA GaT cAT ccT gAT cgA cAA G], 100 μmol/L TaqMan probe [6-FAM-AcA Tcg cAT cgA gcg Agc Acg TAc-TAMRA], 3 mmol/L manganese acetate, 0.5 U AmpErase uracil-N-glycosylase, 7.5 U rTth DNA polymerase, and 10 μl of RNA elution. After initial activation of uracil-N-glycosylase at 50° C. for 2 minutes, RT was performed at 60° C. for 30 minutes, followed by inactivation of uracil-N-glycosylase at 95° C. for 5 minutes. Subsequent PCR amplification consisted of 40 cycles of denaturation at 94° C. for 20 seconds and annealing and extension at 62° C. for 1 minute in an ABI 7700 sequence detector. For each PCR run, negative template and positive template samples were used. The cycle threshold value (Ct-value) is defined as the number of PCR cycles for which the signal exceeds the baseline, which defines a positive value. The sample was considered to be positive if the Ct-value was <50. Results are expressed as genomic equivalents (GE).

### Example 8-Anti-Viral Activity

The results of the testing of the compounds of the invention in the HCV-Huh-5-2 replicon cell assay described above are provided in Table 1 hereunder.

TABLE 3
Antiviral Activities of compounds of the invention
as shown in Tables 1 and 2 on HCV Subgenomic
Replicon Replication in Huh-5-2 Cells

| | Anti-HCV activity^{a} | | |
|---|---|---|---|
| compd | CC₅₀^{b} (μM) | EC₅₀^{c} (μM) | SI^{d} |
| 4a | 90.1 | 26.6 | 3.38 |
| 4b | 44.8 | 11.5 | 3.89 |
| 4c | 36.8 | 6.61 | 5.58 |
| 4d | 66.6 | 15.2 | 4.37 |
| 4e | 74.4 | 15.5 | 4.80 |
| 4f | 27.3 | 9.89 | 2.77 |
| 4g | 41.7 | 3.98 | 10.4 |
| 4h | 41.9 | 15.9 | 2.63 |
| 4i | 26.7 | 3.36 | 7.96 |
| 4j | 11.1 | 3.58 | 3.12 |
| 4k | 44.1 | 2.29 | 19.3 |
| 6b | >101 | 21.2 | >4.77 |
| 6e | 59.8 | 26.3 | 2.28 |
| 7a | 76.4 | 11.1 | 6.88 |
| 7b | 44.8 | 8.28 | 5.42 |
| 7c | 43.1 | 3.88 | 11.1 |
| 7d | 19.8 | 3.14 | 6.33 |
| 7e | 43.1 | 4.12 | 10.4 |
| 7f | 21.9 | 3.33 | 6.59 |
| 7g | >64.8 | 4.08 | >15.9 |
| 7h | 33.1 | 4.35 | 7.62 |
| 16b | 30.3 | 10.9 | 2.78 |
| 16c | 128 | 6.8 | 18.82 |
| 16d | 109 | 2.03 | 53.69 |
| 16e | 94 | 19.6 | 4.80 |
| 19a | 45.2 | 11.7 | 3.86 |
| 19c | 131 | 11.8 | 11.10 |
| 22a | 11.7 | 1.84 | 6.36 |
| 22c | 16.1 | 4.08 | 3.95 |

^{a}Interferon α-2b at 10.000 units/well reduced the signal in the viral RNA (luciferase) assay to background levels; without any cytostatic activity.
^{b}Cytotoxic concentration CC50: concentration required to inhibit the proliferation of exponentially growing Huh-5-2 cells by 50%..
^{c}Effective concentration EC50: concentration required to inhibit luciferase activity in the replicon system by 50%..
^{d}Selectivity index (ratio of CC₅₀ to EC₅₀).

1-22. (canceled)

23. A compound according to the general formula (I), pharmaceutically acceptable salts, solvates, tautomers or isomers thereof, 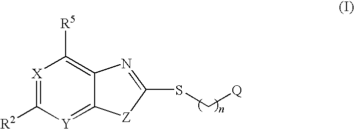
wherein:
X is selected from CR¹ or N;
Y is selected from CR⁶ or N;
Z is selected from NR⁷; O or S;
each of R¹, R², R¹ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8.

24. A compound according to claim 23, wherein X is CR¹ and Y is N, and thereby the compound has a structure according to formula (II); 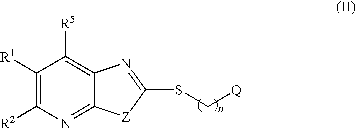
wherein all of R¹, R², R⁵, R⁷, Z, Q and n are as in claim 1.

25. A compound according to claim 23, wherein each of X and Y is N, and thereby the compound has a structure according to formula (III): 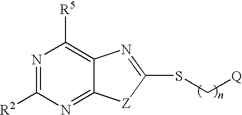
wherein all of R², R⁵, R⁷, Z, Q and n are as in claim 23.

26. A compound according to claim 23, wherein Z is NR⁷.

27. A compound according to claim 23, wherein X is CR¹, Y is CR⁶ and Z is NR⁷, and thereby the compound has a structure according to the general formula (IV), pharmaceutically acceptable salts, solvates, tautomers, or isomers thereof, 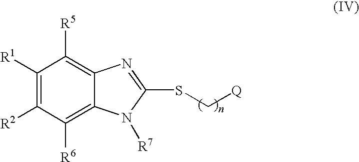
wherein:
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8.

28. The compounds according to claim 23, wherein said substituted aryl or substituted heterocyclic ring are substituted with hydroxy; nitro; alkoxy; a C₁-C₁₆ hydrocarbon group or halogen; wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N.

29. The compound according to claim 23, wherein Q has a structure according to formula (V) 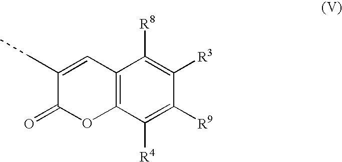
wherein each of R³, R⁴, R⁸ and R⁹ are independently selected from hydrogen; hydroxy; nitro; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N; and n is 1.

30. The compounds according to claim 23, wherein R⁷ is a carbohydrate.

31. The compounds according to claim 23 for use as a medicine.

32. The compounds according to claim 23, for use to treat or prevent a viral infection in a mammal.

33. The compounds according to claim 32, wherein the viral infection is an infection with an RNA virus, more in particular with HCV.

34. A pharmaceutical composition comprising a compound according to the general formula (I), a pharmaceutically acceptable salt, solvate, tautomer or isomer thereof, 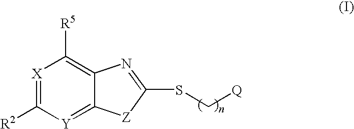
wherein:
X is selected from CR¹ or N;
Y is selected from CR⁶ or N;
Z is selected from NR⁷; O or S;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8,
as an active ingredient in admixture with a pharmaceutically acceptable carrier.

35. A method of prevention or treatment of a viral infection in a mammal comprising administrating a pharmaceutical composition to said mammal, said composition comprising a therapeutically effective amount of a compound according to the general formula (I), a pharmaceutically acceptable salt, solvate, tautomer or isomer thereof, 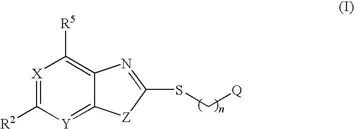
wherein:
X is selected from CR¹ or N;
Y is selected from CR⁶ or N;
Z is selected from NR⁷; O or S;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from substituted or unsubstituted aryl or substituted or unsubstituted heterocyclic ring; and
n is selected from 1 to 8,
as an active ingredient in admixture with a pharmaceutically acceptable carrier.

36. A method for the manufacture of a medicament for the treatment or prevention of a viral infection in a mammal comprising admixing with at least a pharmaceutically acceptable carrier a compounds according to formula (VII), a pharmaceutically acceptable salts, a tautomers, or an isomers thereof 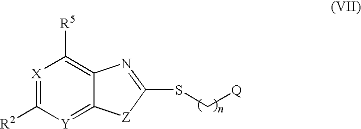
wherein:
X is selected from CR¹ or N;
Y is selected from CR⁶ or N;
Z is selected from NR⁷; O or S;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from hydrogen; substituted or unsubstituted aryl or substituted or unsubstituted heterocyclic ring; and
n is selected from 1 to 8,
provided that Q and R⁷ are not both hydrogen.

37. The method according to claim 36, wherein X is CR¹ and Y is N.

38. The method according to claim 36, wherein each of X and Y are N.

39. The method according to claim 36, wherein Z is NR⁷.

40. A method according to claim 36, wherein X is CR¹, Y is CR⁶ and Z is NR⁷.

41. The method according to claim 36, wherein the viral infection is an infection with an RNA virus, more in particular with HCV.

42. A method of treating or preventing a viral infection in a mammal by using the compounds according to formula (VII), pharmaceutically acceptable salts, tautomers, or isomers thereof 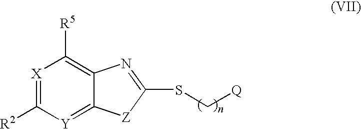
wherein:
X is selected from CR¹ or N;
Y is selected from CR⁶ or N;
Z is selected from NR⁷; O or S;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is selected from hydrogen or a carbohydrate group;
Q is selected from hydrogen; substituted or unsubstituted aryl or substituted or unsubstituted heterocyclic ring; and
n is selected from 1 to 8.

43. A process for the preparation of the compound according to the general formula (I), pharmaceutically acceptable salts, solvates, tautomers or isomers thereof, 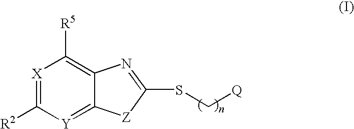
wherein:
X is CR¹;
Y is CR⁶;
Z is NR⁷;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is hydrogen;
Q is selected from substituted or unsubstituted aryl or substituted or unsubstituted heterocyclic ring; and
n is selected from 1 to 8,
comprising the steps of
a) reacting a substituted or unsubstituted 1,2-diamino-phenyl (or phenylenediamines) with CS₂; and
b) coupling the product of (a) with substituted or unsubstituted aryl or heterocyclic ring substituted with halogenalkyl such as 3-chloromethylcoumarins.

44. A process for the preparation of the compound according to the general formula (I), pharmaceutically acceptable salts, solvates, tautomers or isomers thereof, 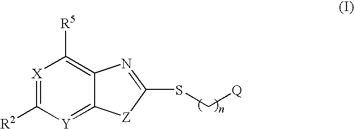
wherein:
X is selected from CR¹ or N;
Y is selected from CR⁶ or N;
Z is NR⁷;
each of R¹, R², R⁵ and R⁶ are independently selected from hydrogen; hydroxy; a C₁-C₁₆ hydrocarbon group or halogen, wherein said hydrocarbon group optionally includes one or more heteroatoms in the main chain, said heteroatoms being selected from the groups consisting of O, S, and N and one or more hydrogen atoms of said hydrocarbon group optionally are replaced by heteroatoms selected from O, S, and N;
R⁷ is a carbohydrate;
Q is selected from substituted or unsubstituted aryl or substituted or unsubstituted heterocyclic ring; and
n is selected from 1 to 8,
comprising the steps of
a) reacting a substituted or unsubstituted 1,2-diamino-phenyl (or phenylenediamines) with CS₂;
b′) coupling the product of (a) with completely protected carbohydrate, such as peracetylpyranose;
c′) reacting the product of step (b′) with substituted or unsubstituted aryl or heterocyclic ring substituted with halogenalkyl such as 3-chloromethylcoumarins; and
d′) if needed, removal of the protecting groups.
